(19) United States
(12) Patent Application Publication
(10) Pub. No.: US 2019/0313966 A1
(43) Pub. Date: Oct. 17, 2019

(54) PAIN LEVEL DETERMINATION METHOD, APPARATUS, AND SYSTEM

(71) Applicant: Somniferum Labs LLC, Las Vegas, NV (US)

(72) Inventor: David Lanzkowsky, Las Vegas, NV (US)

(21) Appl. No.: 15/951,089

(22) Filed: Apr. 11, 2018

(51) Int. Cl. A61B 5/00; A61B 5/16; G06K 9/00

(52) U.S. Cl. CPC A61B 5/4824; G10L 15/01; G06K 9/00315; A61B 5/165

(57) ABSTRACT

An affective system, apparatus and method to analyze pain level states using affective and physiological data collection to determine pain and pain levels in a patient. The system and apparatus includes video cameras to record patient facial or body expressions and movements and combines the recorded expressions and movements with physiological data to determine pain level states in the patient.

### FIELD

This application relates generally to analysis of pain level states and more particularly to using affective data collection to determine pain and pain levels in a patient.

### BACKGROUND

Affective computing is sometimes called artificial emotional intelligence or facial coding and is the study and development of systems and devices that can recognize, interpret, process, and simulate human affects such as facial expression, body gestures and voice tone. It is an interdisciplinary field spanning computer science, psychology, and cognitive science.

Recognizing emotional information requires the extraction of meaningful patterns from the gathered data. In cognitive science and neuroscience, there have been two leading models describing how humans perceive and classify emotion, the continuous and the categorical model. The continuous model defines each facial expression of emotion as a feature vector in a face space, for example, how expressions of emotion can be seen at different intensities. In contrast, the categorical model consists of C classifiers, each tuned to a specific emotion category, to explain why a happy or a surprise face are perceived as either happy or surprise but not something in between.

Many facial expression databases have been created and made public for expression recognition purpose. Two of the widely used databases are CK+ and JAFFE. Defining facial expressions in terms of muscle actions has been used to formally categorize the physical expression of emotions. The central concept of the Facial Action Coding System, or FACS, as created by Paul Ekman and Wallace V. Friesen in 1978 are action units (AU). They are, basically, a contraction or a relaxation of one or more muscles. For example, muscle movement in corners of eyebrows, tip of nose, or corners of the mouth may be indicative of user emotion.

Affective computing was used in the late 1990's to develop a robot head named Kismet by Massachusetts Institute of Technology to recognize and simulate human emotions. Kismet simulates emotion through various facial expressions, vocalizations, and movement. Facial expressions are created through movements of the ears, eyebrows, eyelids, lips, jaw, and head. The Kismet system processes raw visual and auditory information from cameras and microphones. Kismet's vision system can perform eye and motion detection.

### SUMMARY

Analysis of pain levels of patients as they interact with a diagnostic system may be performed by gathering data from measuring facial expressions, head and body gestures, speech analysis and physiological conditions. This is done using machine learning techniques that process different user characteristics such as speech recognition, natural language processing, or facial expression detection. Detecting affective information begins with sensors which capture data about the user's physical state or behavior without interpreting the input. For example, a video camera captures facial expressions, body posture, and gestures, while a microphone captures speech.

Pain level analysis may be used to inform health care professionals of the pain level currently being experienced by a patient. The diagnostic system collects data from an individual while the individual interacts with the diagnostic system which may or may not include human health care professionals and a robotic system. The data collecting may further comprise collecting one or more of speech, facial data, physiological data, and body/head movement data from an accelerometer or other sensor. A webcam may be used to capture one or more of the facial data and the physiological data. Other sensors detect emotional cues by directly measuring physiological data, such as skin temperature and galvanic resistance. The method may further comprise inferring pain levels based on collected data. The collected data may be compared to data recorded when the patient was not experiencing pain to assess the comparative pain level.

Data is collected as a patient interacts with a diagnostic system which may include a patient intake robot. Data including facial expression, body language and speech recognition may be detected and collected by the system and the robot. Analysis is performed on this data and evaluated against parameters to determine the pain metrics of the patient. The diagnostic system may also include physiological measurement technology including medical imaging techniques such as Iris Recognition Technology (IRT) and Computerized Axial tomography (CAT) in combination with facial, head/body movement and speech recognition technology. A functional MRI (Magnetic Resonance Imaging) and Galvanic Skin Response measuring unit may also be employed in some embodiments.

In some embodiments, certain biomarkers such as those in sweat or blood could be used and there are simple devices for measuring analytes. Salivary cortisol, α-amylase (sAA), secretory IgA (sIgA), testosterone, and soluble fraction of receptor II of TNFα (sTNFαRII) serve as objective pain measures. Blood biomarkers may also be used but this requires invasive techniques and may cause pain which may affect the pain algorithm.

Speech recognition may be sensed by microphones and recorded and analyzed for observed changes in speech characteristics including tone, tempo, and voice quality to distinguish emotions. The sensed speech may be compared to a baseline speech pattern recorded when the patient was not experiencing pain to assess the perceived pain level.

The detection and processing of facial expression are achieved through various methods such as optical flow, hidden Markov models, neural network processing or active appearance models. One or more techniques can be utilized or they can be combined (e.g. facial expressions and speech, facial expressions and hand gestures, or facial expressions with speech) to provide a more robust determination of the patient's pain level state.

In embodiments, a computer implemented method for detecting patient pain levels may comprise: collecting facial expression, body language and/or speech recognition data; combining the collected data with physiological measurement technology data from an individual; analyzing the collected data to determine pain state information; and communicating the pain level information with a health care provider. In some embodiments, the method may include displaying the pain level information in a graphic or numerical visualization.

In some embodiments, a computer program product stored on a non-transitory computer-readable medium may comprise: code for collecting facial expression, body language and/or speech recognition data while the individual interacts with a robotic or other diagnostic system; code for analyzing, using a web services server, the facial expression, body language and/or speech recognition data with, optionally, physiological data to produce pain state information; and code for displaying or communicating pain state information.

In some embodiments, a computer system may comprise: a memory for storing instructions; one or more processors attached to the memory wherein the one or more processors are configured to: collect facial expression, body language and/or speech recognition data as well as physiological data of an individual while the individual interacts with a diagnostic system; analyze, using a web services server, the facial expression, body language and/or speech recognition data to produce pain level state information; and communicate or display the pain level information.

A pain level diagnostic system may include computer software and hardware in combination with various sensors including facial recognition, speech and head/body movement as well as physiological measurements including: pulse and heart rate, blood volume pulse, galvanic skin response, and facial electromyography.

### BRIEF DESCRIPTION OF THE DRAWINGS

The following detailed description of certain embodiments may be understood by reference to the following figures wherein:

FIG. 1 is a flow diagram of a method for detecting patient pain levels;

FIG. 2 is a diagram showing patient interaction with an embodiment of a diagnostic system

FIG. 3 is a diagram of another embodiment of a diagnostic system;

FIG. 4 is diagram illustrating a diagnostic network;

FIG. 5 is a diagram showing a health care professional interacting with a diagnostic network; and

FIG. 6 is a showing patient interaction with a diagnostic network.

### DETAILED DESCRIPTION

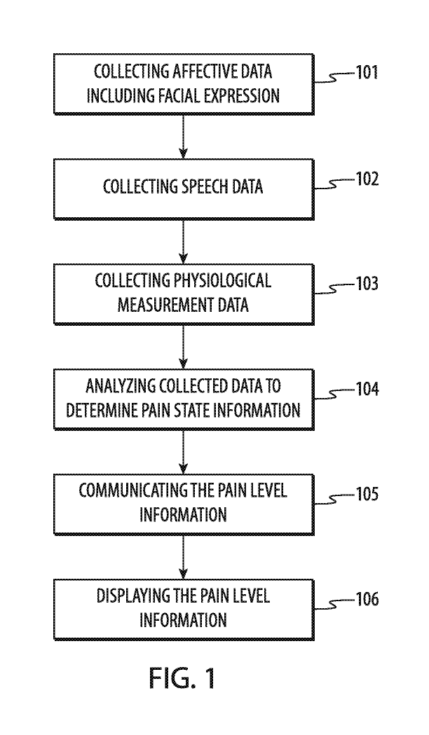

Reference will now be made to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims. Like reference numerals denote like structure throughout the various embodiments disclosed herein with reference to FIGS. 1-6. However, those skilled in the art will readily appreciate that the detailed description given herein with respect to these figures is for explanatory purposes only and should not be construed as limiting.

Various embodiments disclosed herein are directed toward addressing one or more of the problems discussed above, while prioritizing the patient's health, safety, choice of treatment, reduced adverse effects, and general best interests. An optimal pain treatment plan will have the added benefits of improvements in social and legal issues for the patient. The present disclosure provides a description of various methods and diagnostic systems for analyzing patient pain level state as the patient interacts with the diagnostic system.

Observing, capturing, and analyzing the affective data gathered can yield significant information about patient pain level states. Analysis of the pain level states may be provided by web services and thus allow treatment to be prescribed. With the disclosed methods and systems, health care professionals may objectively determine the pain levels that patients are experiencing. Affect data can be communicated across a distance and thus pain levels of patients in distant locations may be remotely diagnosed by health care professionals.

Affective data may include facial analysis for expressions such as smiles or brow furrowing. Body gestures could also be efficiently used as a means of detecting a particular pain level state of the user, especially when used in conjunction with speech and facial analysis. Depending on the specific action, head/body gestures could be simple reflexive responses, like lifting of the shoulders or moving or nodding one's head. Two different approaches in gesture recognition may be used: a three dimensional model; and an appearance-based model. The three dimensional model uses information from key elements of the body parts in order to obtain several important parameters, like palm position or joint angles. An appearance-based system uses images or videos from the diagnostic system for direct interpretation. As used herein affective data may also include speech and/or physiological data.

Physiological monitoring could also be used to detect a user's pain level state by monitoring and analyzing a patient's physiological signs. These signs may include pulse and heart rate, blood volume pulse, galvanic skin response, facial electromyography that may be combined with speech and facial recognition and head/body movement to assess a patient's perceived pain level. A patient's blood volume pulse (BVP) can be measured by a process called photoplethysmography, which produces a graph indicating blood flow through the extremities. When the patient is stimulated by the diagnostic system, the heart usually ‘jumps’ and beats quickly for some time, causing the amplitude of the cardiac cycle to increase. As the patient calms down, and as the body's inner core expands, more blood flows back to the extremities, and the cycle will return to normal. Another BVP measurement technique may include infra-red light shone on the patient's skin by special sensor hardware, wherein the amount of reflected light is measured. The amount of reflected and transmitted light correlates to the BVP as light is absorbed by hemoglobin found in the blood stream.

Facial electromyography may also be used as a data input to measure a patient pain level. Facial electromyography is a technique used to measure the electrical activity of the facial muscles by amplifying the tiny electrical impulses that are generated by muscle fibers when they contract. The corrugator supercilii muscle and zygomaticus major muscle are the two main muscles used for measuring the electrical activity in facial electromyography. The corrugator supercilii muscle, also known as the ‘frowning’ muscle, draws the brow down into a frown, and therefore is the best test for negative, unpleasant emotional response including possible pain indication. The zygomaticus major muscle is responsible for pulling the corners of the mouth back when smiling, and therefore is the muscle used to test for a positive emotional response which may be a contraindication of pain.

Galvanic skin response (GSR) is a measure of skin conductivity, which is dependent on how moist the skin is. As the sweat glands produce this moisture and the glands are controlled by the body's nervous system, there is a correlation between GSR and the arousal state of the body. The more aroused a subject is, the greater the skin conductivity and GSR reading. Galvanic skin response (GSR) may be included in the diagnostic system to indicate an excited or aroused state. At low levels of excitement, there is a high level of resistance recorded, which suggests a low level of conductivity and therefore less arousal. This is in clear contrast with the sudden trough in recorded resistance where the patient is experiencing pain because the patient is very stressed and tense. GSR uses electrodes placed on the patient's skin and then applies a small voltage between them. The conductance is measured by a sensor. To maximize comfort and reduce irritation the electrodes can be placed on the torso, legs or feet, which leaves the patient's hands fully free to interface with a keyboard and mouse or other elements of the diagnostic system.

In some embodiments, aesthetically pleasing and displeasing images may be shown to the patient in the diagnostic system to measure the patient response to further gauge the patient pain level. Similarly, haptic stimulation could be used to impart unpleasant physical sensations to the patient (e.g. hands, arms, legs) and thus gauge the pain level by the measured response as sensed by the diagnostic system.

Speech recognition technology may be used in conjunction with facial affect technology to assess the patient pain level. For example, parameters such as: changes in the frequency of the patient voice; high/low pitch of the voice; frequency change over time (e.g. rising, falling or level); pitch range—difference between the maximum and minimum frequency of an utterance; speech rate of words or syllables uttered over a unit of time; and stress frequency—measures the rate of occurrences of pitch accented utterances. Other voice parameters indicative of pain level include: breathiness (aspiration noise in speech); high or low frequencies; loudness speech amplitude; pause transitions between sound and silence; and discontinuity between pitch frequency transitions.

In some embodiments, a system such as the PROMIS (Patient Reported Outcomes Measurement Information System) developed by the National Institute of Health (NIH) may be used. This system asks patients to provide quantitative pain intensity estimates by answering questions either interactively through a computer or using paper questionnaire. In some embodiments, the questions are asked by a robot or medical professional in conjunction with the affective measurement embodiments disclosed herein to provide a more objective determination of pain levels.

FIG. 1 is a flow diagram of a method for detecting patient pain levels using pain level diagnostic system which may be computer implemented. Various operations in the method shown in FIG. 1 may be changed in order, repeated, omitted, or the like without departing from the disclosed embodiments. Referring to FIG. 1, in operation 101 the system collects affective data including facial expression, body language and/or other affective data from the patient. Operation 102 includes optionally collecting speech data from the patient. Operation 103 includes optionally collecting physiological measurement data from the patient. Operation 104 includes combining and analyzing collected data to determine pain state information. Operation 105 includes the operation of communicating the pain level information to a health care provider, patient, or system. Operation 106 includes the optional step of displaying the pain level information to a health care provider and/or to the patient in a graphic or numerical visualization.

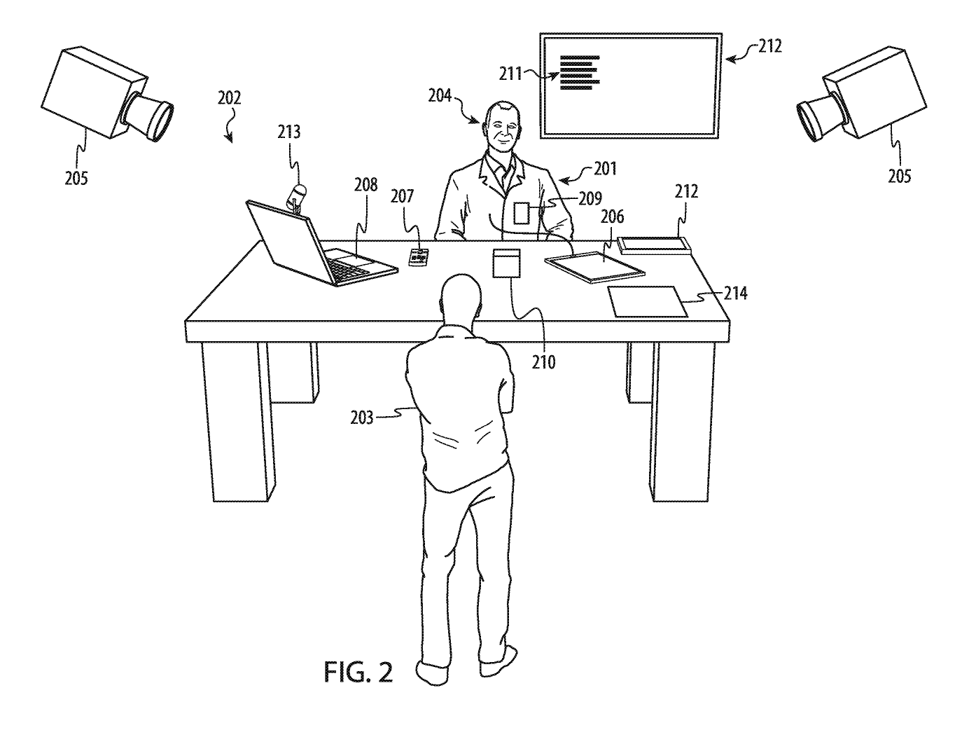

FIG. 2 further illustrates operation 101 for collecting data including patient interaction with a computer implemented pain level diagnostic system. Referring to FIG. 2, a patient 201 interacts with diagnostic system generally shown at 202 which collects pain recognition data from a patient 201 while the patient is presented in front of the diagnostic system 202. Patient 201 may be standing or seated adjacent the diagnostic system 202. In some embodiments, a robot or a health care professional 203 may interact with patient 201 to assist in obtaining pain recognition data. In one embodiment, the patient could interact with the health care professional or robot which administers the PROMIS system as discussed above. Based on facial expression analysis the patient's face 204 may reflect a level of pain ranging from, in one embodiment, 0-10, with 10 being extreme pain and 0 representing low or no pain.

Referring again to FIG. 2, the collecting of pain level state data may further comprise collecting one or more of facial data, physiological data, and movement data. In some embodiments, one or more webcam or video cameras 205 may be used to capture one or more of the facial data. In some embodiments, other sensors 206 such as galvanometers, photoplethysmography, electromyography and accelerometers may be connected to or otherwise associated with patient 201 and used to collect the physiological data in optional operation 103. Alternatively, the data may be collected and/or stored by a peripheral device such as a handheld portable phone 207 or a computer 208 through Skype or similar visual systems to allow remote pain diagnosis. In some embodiments, the physiological data and actigraphy data may be obtained from one or more biosensors 209 including wireless or wired connections attached to patient 201. For example, in some embodiments, biosensors 209 could include one or more of a body position sensor, a sound generator, a snore or apnea sensor, a spirometer, a glucometer sensor, pulse oximeter, blood pressure sensor, galvanic skin response unit, airflow sensor, electrocardiogram sensor, electromyogram sensor, and temperature sensor to monitor the patient's medical status.

Referring again to FIG. 2 patient 201 may interact with system 202. In one embodiment patient 201 interacts with the PROMIS system using a keyboard, a mouse, a controller, a remote, a motion sensor, a camera sensor, or similar device 210. In some embodiments, patient utilizes a paper questionnaire 214. The answers given by patient 201 using the PROMIS system may be correlated with affective data gathered by system 202 to provide additional pain measurement data. As the patient interacts with the system 202, the pain level states 211 of the patient 201 may be displayed, observed and/or analyzed on a display device 212. The facial data of the patient may be captured by one or more webcams, video camera, or other camera devices 205. Facial data obtained from webcam 205 may include facial actions and head gestures from head 204 which may in turn be used to infer pain level states. In some embodiments, a microphone or other sound capture device 213 may be used to capture the speech from patient 201 in response to prompts or contemporaneously throughout the testing.

In optional operation 103, the pain level states may also be captured using one or more biosensors 209. The biosensor 209 may be attached to patient 201 to capture information on electrodermal activity (EDA) or skin conductance or galvanic skin response (GSR), accelerometer readings, skin temperature, heart rate, heart rate variability, and other types of physiological analysis of an individual. The video and physiological observations may be performed and analyzed locally. Alternatively, the video and physiological observations may be captured locally on a diagnostic system machine or computer 208 with analysis being performed on a remote server machine. A functional MRI (Magnetic Resonance Imaging) and Galvanic Skin Response measuring unit 212 may also be employed in addition to or in conjunction with biosensor 209 in some embodiments.

In some embodiments, stimuli may be provided in conjunction with administration of the PROMIS system. For example, electrical, thermal, light, pressure or electromagnetic stimulation may be provided to the patient at various levels through or by sensors 206 or 209 to measure patient affective or sensed pain response to those stimuli. For example, slight electrical shock, applied heat, intense light, squeezing of a finger or other body part or other stimulation could be provided to the patient and the patient facial affective reaction as well as sensed physiological response could be measured. In particular, nociceptive and neuropathic pain response may be measured in response to such stimuli.

The PROMIS system may also be used to measure both nociceptive and neuropathic pain. Nociceptive pain may be described by terms such as “achy, deep, sore, and tender” while neuropathic pain may be described as “numb, tingly, stinging or electrical” types of feeling. All of these sensed pain reactions and PROMIS descriptions from patient 201 may be correlated with affective data compiled by system 202 to provide data to researchers or to the health care professional or robot 203 in diagnosing the pain level state of patient 201.

In some embodiments, certain biomarkers such as those in sweat or blood could be obtained by sensor 206 and biosensor devices 209 for measuring analytes. For example, salivary cortisol, α-amylase (sAA), secretory IgA (sIgA), testosterone, and soluble fraction of receptor II of TNFα (sTNFαRII) serve as objective pain measures. It should be understood that blood biomarkers require invasive techniques and may cause pain which may affect the pain algorithm. Blood samples may thus be taken before or after the patient interaction with the system 202 to minimize the impact of this invasive procedure. Affect sensing may include collecting one or more of facial, physiological, and accelerometer data. The physiological data analysis may include electrodermal activity or skin conductance, skin temperature, heart rate, heart rate variability, respiration, and other types of patient analysis measured through sensors 206 and biosensors 209.

FIG. 2 shows image capture during diagnostic system testing. System 202 may capture patient facial response by cameras 205 with or without a visual rendering displayed on computer 208 or display 212. The data from facial expression 204 may include video and collection of information relating to pain level states. In some embodiments, webcam 205 may capture video of the patient face 204 and body movement from patient 201 including capturing a patient's interaction with the system 202 including video of the patient. As discussed above, in some embodiments, video could be captured while patient 201 interacts with the PROMIS system. Webcam 205, as the term is used herein may refer to a webcam, a camera on a computer (such as a laptop, a net-book, a tablet, or the like), a video camera, a still camera, a cell phone camera, a mobile device camera (including, but not limited to, a forward facing camera), a thermal imager, a CCD device, a three-dimensional camera, a depth camera, and multiple webcams used to capture different views of patients or any other type of image capture apparatus that may allow image data captured to be used by the electronic system.

The console display associated with computer 208 may include any electronic display, including but not limited to, a computer display, a laptop screen, a net-book screen, a tablet computer screen, a cell phone display, a mobile device display, a remote with a display, a television, a projector, or display such as display 212. Computer 208 may also include a keyboard, mouse, touchpad, wand, motion sensor, and other input means 210. In some embodiments, video is captured by webcam 205 while in others a series of still images are captured.

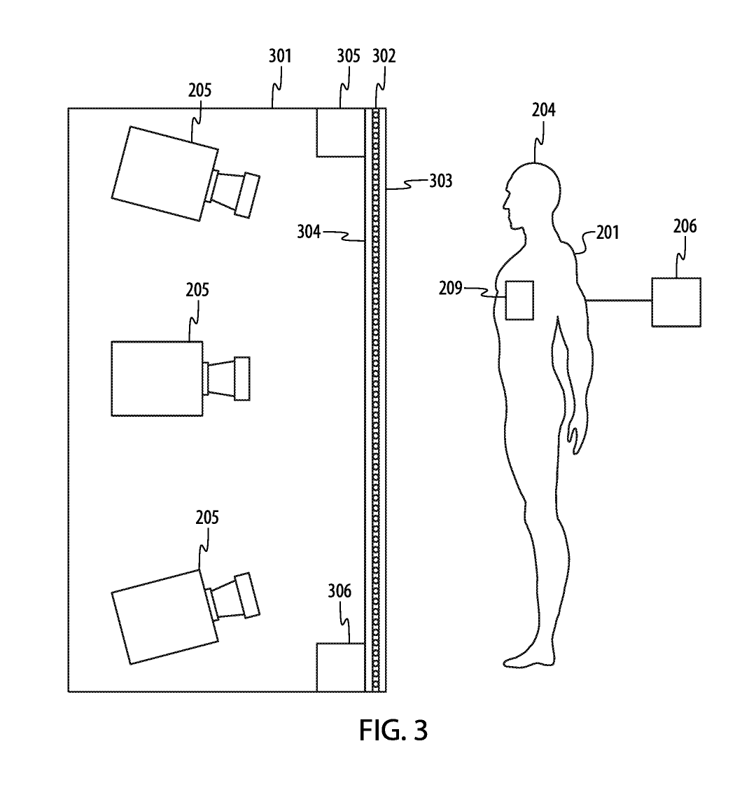

Referring to FIG. 3, in some embodiments, analysis of facial expression 204, hand/body movement or gestures of patient 201, and physiological data obtained by sensors 206 and/or biosensors 209 may be accomplished using the captured images of patient 201 presented in front of an automated or robotic apparatus 301. The visual images from cameras 205 may be used to identify smiles, frowns, and other facial indicators of pain level states. The gestures of patient 201, including head gestures, may indicate certain levels of pain. For example, a head gesture of moving toward or away from camera 205 in response to certain stimuli or instruction by robotic system 301 may indicate increased or decreased levels of pain.

Referring again to FIG. 3, apparatus 301 could include a one way screen 302 which allows certain images to be shown to patient 201 on front 303 while cameras 205 record patient affective or physiological data through the backside 304 of screen 302. Cameras 205 may or may not be visible to patient 201 through one way screen. Analysis of those captured images, and sensed physiology may be performed. Determination of pain level states may be performed based on the analysis of information and images which are captured. The analysis can include facial analysis and analysis of the head gestures. The analysis can include analysis of physiology including heart rate, heart variability, respiration, perspiration, temperature, and other bodily evaluation as measured by sensors 206 and 209.

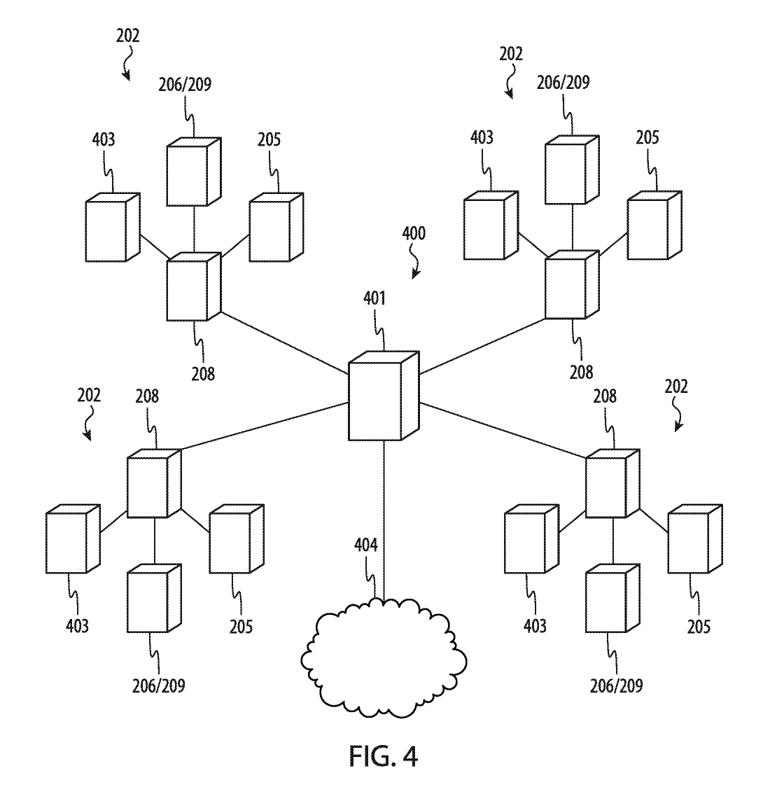

Screen 302 may be a touchscreen to allow patient 201 to respond to questions or provide input to apparatus 301. For example, PROMIS questions could be displayed on screen 302 and patient 201 may respond to those questions. In some embodiments, data from sensors 206/209 may be displayed on screen 302 for patient viewing. In some embodiments, stimuli as discussed above may be provided to patient through sensors 206/209 and patient response to those stimuli may be recorded by the system. In some embodiments, a sound capture device 305 such as a microphone may be included within or outside of apparatus 301 to allow vocal sounds from patient 201 to be sensed and recorded. A controller 306 may be included as part of apparatus 301 to record and analyze affective, speech, and physiological data sensed and recorded by apparatus 301. Controller 306 may be connected to a network such as shown in FIG. 4 either through wired or wireless connection. Patient pain level states may be displayed onscreen 302 to be viewed by patient 201 in some embodiments.

Referring again to FIG. 2 as the patient 201 interacts with the system 202, patient 201 has a sensor 206 and or 209 attached to or associated with him or her. The sensor 206/209 may be placed on or attached to the wrist, palm, hand, head, or other part of the patient body 201. The sensor 206/209 may include detectors for electrodermal activity, skin temperature, and accelerometer readings. Other detectors such as heart rate, blood pressure, EKG, EEG, further brain waves, and other physiological detectors may be included. The sensor 206 may transmit information collected to a receiver using wireless technology such as Wi-Fi, Bluetooth, 802.11, cellular, or other bands. The receiver may provide the data to one or more components 208 in the diagnostic system 202. In some embodiments, the sensor 206/209 will save various physiological data in memory for later download and analysis by system 202. In some embodiments, the download of data can be accomplished through a USB port from sensor 206/209 or computer 208.

Electrodermal activity may be collected continuously or on a periodic basis by sensor 209 in some embodiments as the patient 201 interacts with the diagnostic system 202. The electrodermal activity may be recorded to a disk, a tape, onto flash memory, into a computer system 208, or streamed to a server. The electrodermal activity may be analyzed to indicate indication of pain level states based on changes in skin conductance. Skin temperature may be collected on a periodic basis by sensor 209 or an as needed basis and then be recorded. The skin temperature may be analyzed and may indicate pain level states based on changes in skin temperature.

Accelerometer data may be collected and indicate one, two, or three dimensions of motion by sensor 206. The accelerometer data may be recorded. The accelerometer data may be analyzed and may indicate pain level states based on patient voluntary or involuntary movement in response to electromagnetic or other stimuli or without external stimuli.

In some embodiments, multiple sensors 209 may be attached to a patient. In embodiments, the sensors could be attached to each wrist and each ankle of patient 201 to detect motions and relative positions of the arms and legs. A sensor could also be attached to the head 204 or elsewhere on the body of patient 201. In embodiments, the sensor 209 could be used to evaluate motions for patient responses to external stimuli. In embodiments, the sensors could be used to evaluate body position. Further, sensors 206 and 209 could be used to evaluate both motion and emotion.

Referring to FIG. 4, the operation 104 of combining the collected affective, speech and physiological data includes analyzing, using a web services server 401 connected directly or wirelessly to one or more diagnostic systems 202 in a network 400. Server 401 may also be connected to the internet or cloud 404 wirelessly or by wired connection. It should be understood that computers 208 may be directly or wirelessly connected to cloud 404 without being connected to server 401. Multiple systems 202 may be used for comparative purposes or in a research environment to compare pain state level data from one or more patients. The data collected in operations 101, 102 and 103 is processed to produce pain level state information. The web services server 401 may include remote computer 208 from, or part of, the diagnostic systems 202. Computer 208 may provide sensed information data to server 401 and the analyzing operation 104 can include aggregating the sensed data information in and comparing it to previous sensed data from the same patient.

While sensed data may be raw data, the information may also include information derived from the raw data. The sensed data information may include all of the data or a subset thereof. The information may include information on the pain level states experienced by the patient or other patients using other systems 202 or to previous measurements to allow comparative pain level determinations and measurements to be made. The pain level information may include assessing pain level states based on the data which was collected. The assessed pain level states may include one or more of numerical or graphic representations of patient pain level states. Analysis of the pain level state data may take many forms and may be based on comparison to the patient or may include comparisons to known pain levels in a plurality of patients. As described herein system 202 could include connection to the PROMIS system and may provide data to researchers or to health care professional to improve the PROMIS measurement system.

In some embodiments, some analysis may be performed on a diagnostic system computer 208 in system 202 before that data is uploaded to server 401 while some analysis may be performed on server 401. Various embodiments of the disclosed embodiments may include a computer program product embodied in a non-transitory computer readable medium that includes code executable by one or more processors in computer 208 or server 401. In one embodiment, a controller unit 403 may execute instructions and carry out operations associated with a diagnostic system 202 as described herein. Using instructions from device memory, controller 403 may regulate the reception and manipulation of input and output data between sensors 206/209, cameras 205 and other components in the diagnostic system 202. Data transferred to or from the device may be encrypted to comply with HIPAA or other regulations. Controller 403 may be implemented in a computer chip or chips. Various architectures can be used for controller 403 such as microprocessors, application specific integrated circuits (ASIC's) and so forth. Controller 403, together with an operating system may execute computer software code and manipulate data. The operating system may be a well-known system or a special purpose operating system or other system as may be known or used. Controller 403 may include memory capability to store the operating system and patient or other data. Controller 403 may also include application software to implement various functions associated with the portable electronic device. For example, an algorithm may be programmed into controller 403 to guide a patient through various operations as part of the diagnostic system 202 pain level evaluation.

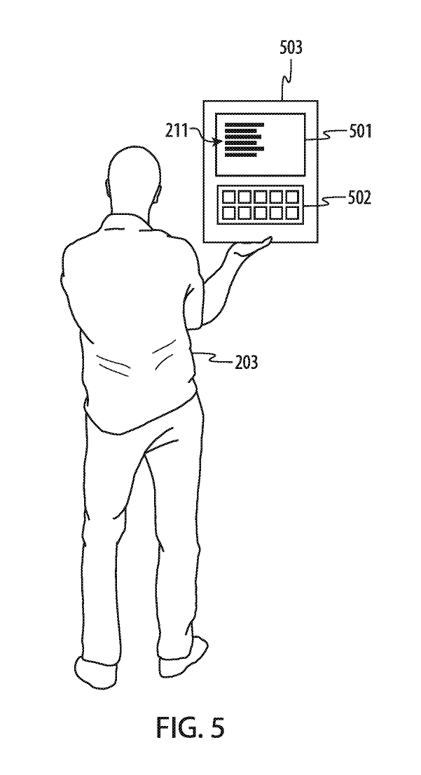

Referring to FIG. 5, in some embodiments, the pain level information 211 of the patient 201 is communicated directly or remotely to a health care professional 203 in operation 105. Information 211 may be displayed on a wired or wirelessly connected display 501. Display 501 can be associated with a computer, tablet wireless telephone or other electronic device 503. Health care professional 203 may input data or analysis on a keyboard 502 or other input device associated with display 501 Likewise, the pain level states of the patient may be presented to the patient him or herself. The pain level state information 211 of the patient may be presented through a set of color representations, or through a graphical representation Likewise the pain level state information 211 may be represented in one of a group selected from a bar graph, a line graph, a numerical representation, a smiling face, a frowning face, and a text format or the like.

Communication of pain level can be done in real time while the patient is being monitored as shown in FIG. 2 or remotely as shown in FIGS. 3, 5 and 6. In some embodiments, the various sensory inputs to the patient can be modified based on this real time affect communication in order to test patient reliability (is the patient faking pain?). Health care professional 203 may input prompts or initiate stimuli through keyboard 502. Alternatively, communication of pain level information 211 can be after the sensing is completed or after a specific session or test is completed. The pain level information 211 can be communicated as a single graphical representation which could be a set of icons or other symbol that can connote positive or negative rating. The affect can also be communicated numerically, with the number indicating a numerical pain level. For example, a patient pain level could be expressed as a percentile as in “The patient pain level correlates to the 61st percentile of patients in a selected demographic or to patients overall”. The image of the patient 201 may also be displayed on display 501 either in real time or as a collection of images.

In some embodiments, pain level information 211 can be communicated to the health care professional 203 along with the reaction of the patient to the sensory measurement and input from sensors 206/209. The patient reaction can include the response to external stimuli administered to the patient such as small electrical jolt or other stimuli through sensors 206 or biosensors 209 on or otherwise connected to, or associated with, the patient 201. The patient's reaction to the diagnostic sensing can be used to recommend changes to pain relief medication or to dosing of a particular medication and could be used to recommend medication or doses to others by comparing data to other patients using the diagnostic system.

In some embodiments, in operation 106, the pain level information may be displayed on a monitor 212 real time with the patient present as in FIG. 2 or remotely as in FIG. 3, 5 or 6 to allow the data to be analyzed and presented in summary form or in real time format to health care professional or in printed graphical or numeric form 211 and provided to a health care professional 203 or to the patient 201 in a visualization. For example, referring to FIG. 2, the information 203 can be presented on a display 212. The pain level state information 211 may be graphical or textual presentation of the information. The visualization may be presented to and used by a health care professional 203 remotely as in FIG. 5 or live as in FIG. 2 to identify how the patient 201 is reacting to pain relieving medications or doses. Alternatively, the visualization 211 may be used by health care professionals to better understand the patient's reaction to the medication or a various doses thereof. Optimal medication and dosing levels could be included based on the visualization 211.

Referring again to FIG. 4, communicating pain level state information to and through a health care network 400 may be accomplished using one or more diagnostic systems 202. The health care network 400 may comprise a research or patient care community and the identity of the individual patient could be masked to preserve patient privacy while allowing the data to be shared and compared to other patients on the network 400. People on the health care network 400 who receive the pain level state information may themselves be patients. In some cases, however, the people on the health care network who receive the pain level state information may be health care professionals and not patients themselves or at least not involved with the diagnostic system with which the patient is interacting. Certain health care professionals may only be interested in the individual patient and any activities or reactions of the individual patient while others may be interested in the collective data assembled by one or more systems 202.

The sharing of pain level states could replace or augment other subjective pain level systems. The pain level state data for the patient could be used to augment a subjective pain level rating system. Alternatively the person's affect could replace and be used as the only pain level rating system. In some embodiments, affect data could be shared across a network 400 which indicates the level of pain for the individual and may be compared to other patients. For example, in a pain network 400 the health care professionals on the network could see how pain level varies with certain stimuli or sensed data. In some embodiments, pain level state information may be shared across a research or clinical network based on the pain level states.

It will be understood that throughout this disclosure, that while a reference may be made to an individual or a person with respect to sensing and data collection, analysis, displaying, and the like that the data could be shared anonymously and apply equally to various individuals or groups across a network 400 such as the Internet. All such embodiments for both groups and individual patients fall within the scope of this disclosure.

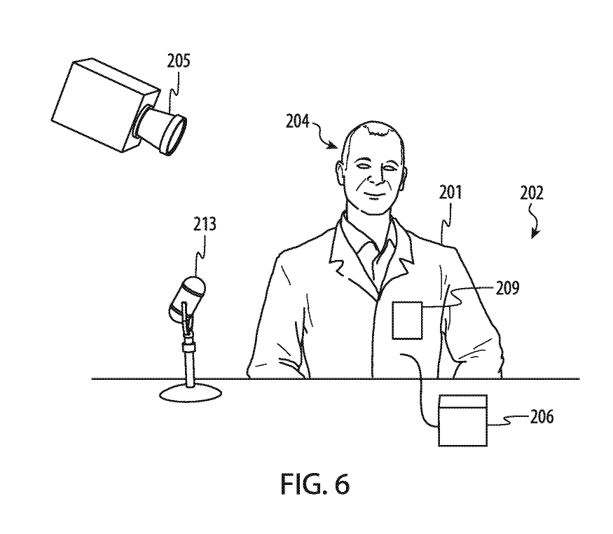

FIG. 6 shows diagnostic network 202 interacting with a patient 201 by collecting and recording facial expressions, such as smiles or brow furrows from video observations of a patient head and face 204 by camera 205. Physiological data may be obtained by sensors 206 and biosensors 209 connected to or otherwise associated with patient 201. For example, heart rate, heart rate variability, autonomic activity, respiration, and perspiration may be observed from sensors 206 and 209. In some embodiments, biosensor 209 may be used to capture physiological information and may also be used to capture accelerometer readings sensing patient movement either in response to stimuli or to verbal input. In some embodiments, data collection and pain level state analysis may be performed in a single step. Additionally, in some embodiments, the analyzing of the pain level state information may be analyzed along with known data to correlate the pain level state information of the patient with the known pain level data. The analyzing of pain level state data may include inferring of pain level states for the patient as they interact with the diagnostic system 202. The analyzing may be performed locally on a client computer system 208 as in FIG. 2. The analyzing may also be performed on a server computer 401 or other remote system as in FIG. 4. In some embodiments, patient speech may be recorded through a microphone 213 to determine the speech patterns of patient 201. These recorded or sensed audio signals may be communicated to the network or computer system 208 and combined with visual and physiological data to determine patient pain level states.

In some embodiments, the interaction may include a patient option of electing, by the individual patient 201, to share (anonymously or not) the pain level state information across a network 400 to gauge relative pain level determination with other patients similarly situated. For example, patients with a certain disease such as lung cancer may elect to have their data compared with other lung cancer patients to experience piece of mind that their pain levels are not out of the expected norm from those similarly situated. There may be a stage where the individual can opt in to sharing of pain level states in general, only for a specific purpose, or only for a specific session. In embodiments, the individual may elect to share the pain level state information after a diagnostic session is completed. In other embodiments, the sharing may be real time so that the patient experience and reactions may be modified real time as the individual patient is interfacing with the diagnostic system 202. In some embodiments, when a patient elects to share pain level states the pain level state information may be modified. For example, a patient may choose to share a pain level state which is more positive at certain times than the inferred less positive pain level states which were analyzed.

In some cases, the process may include the operation 106 of displaying the affect or pain level state from the individual to a health care professional or others who are involved in the pain diagnostic environment as shown in FIG. 2 and FIG. 5. The display of pain level information 211 may be represented through a set of color representations, through a graphical representation, a set of thumbnails, or through a text communication.

In some embodiments, operation 101 includes collecting images of the patient while the patient is interacting with the diagnostic system 202. These images may be video or may be individual still photographic images from cameras 205. The images may be standard visual light photographs or may include infrared or ultraviolet images. In some embodiments, the flow includes posting an image from a session within the diagnostic network 400. The image may include a facial expression. A group of images may be included as a set of thumbnails. A facial expression may be selected because it is a typical facial expression or because pain levels are experienced. In some embodiments, the image posted may include a video of the whole patient or only face 204. The images posted can assist the health care professional in diagnosing pain levels and may assist the health care professionals in creating a research database of pain level information.

Based on the pain level states of the individual, a recommendation to treat the pain level of the patient may be provided by the health care professional. The flow may include recommending certain medication or course of treatment, based on the pain level state information, to the patient. A recommendation may include recommending a particular health care professional experienced in certain pain levels based on the pain level state information. A health care professional may be recommended based on skill, education, or experience. A correlation between an individual patient and health care professionals may be based on the correlation and the pain level states of the individual patient.

One or more recommendations may be made to the patient based on the pain level states of the individual. A medication or course of treatment may be recommended to the individual based on his or her pain level states as determined by the pain level diagnostic system. A correlation may be made between the individual patient and other patients with similar pain level state exhibited during similar diagnostic system testing. Likewise a movie, video, video clip, from camera 205 or display screen 302 or other communication from system 202 may be provided to individual patients 201 based on their determined pain level state.

The diagnostic system may include the hardware for performing the affect sensing. In other embodiments there may be a separate device, such as a laptop, personal computer, or mobile device which captures data associated with the affect sensing. The output of the affect sensing can be forwarded for analysis to the diagnostic network 400. The web services can be part of a diagnostic system. Alternatively, the web services can be a separate analysis system which provides input to the diagnostic system 202. The web services may be a server or may be a distributed network of computers as shown in FIG. 4.

In some embodiments, some analysis may be performed by the diagnostic system 202. In other embodiments, the diagnostic system 202 apparatus collects data and the analysis is performed by the web services in network 400. In some embodiments other patients may be interacting with other terminals on the diagnostic network 202 along with the patient who is having his or her pain level state sensed. In embodiments, each of the patient and the other patients will have their pain level sensed and anonymously provided to the web services to be compared for relative pain analysis.

Analysis of the affect and other data is performed by the diagnostic system analysis server 401 or locally in a system 202. The diagnostic system analysis module may be part of the diagnostic system 202, part of the web services, or part of a computer system that provides an analysis engine. The facial, physiological, and speech data may be analyzed along with the patient information for context. Based on this analysis the pain level may be determined and a treatment regimen prescribed. An aggregating engine will compile and analyze the sensed data from the patient and possibly from the other patients. The aggregating engine can be used to assess pain levels based on the combined data sensed from all of the patients involved. In some embodiments, the aggregating engine may gather other sources of information for aggregation including research or other data. In some embodiments, the pain level states may be modified based on the aggregation of all this information.

A graphical representation of pain level state analysis may be shown for diagnostic analysis and may be presented on an electronic display such as display 212 or 501. The pain level analysis may be used to identify pain levels and recommend treatment where indicated. The display may be a television monitor, projector, computer monitor (including a laptop screen, a tablet screen, a net-book screen, and the like), a cell phone display, a mobile device, or other electronic display. An example window is shown in displays 212 and 501 which include, for example, a rendering of pain level state information 211. A patient or health care provider may be able to select among a plurality of visual renderings using various buttons and/or tabs such as on input 502. The user interface allows a plurality of parameters to be displayed as a function of time, synchronized to the patient pain level states.

The visual representation 211 displays the aggregated pain level state information. The pain level state information may be based on a demographic basis for those patients who comprise that demographic. In some embodiments, the pain level state information may be illustrated as a percentile of all patients or patients similarly situated. For example, a patient could be determined as being in the 73rd percentile (0-100 scale) based upon the source of pain or based upon patients similarly situated such as those with arthritis, broken bones, cancer etc. A 73rd percentile ranking, for example, could mean that the subject patient is experiencing more pain than 73% of tested patients. Thus, in this example, display 212 or 501 would illustrate by bar graph or numerically a 73 score.

The various demographic based graphs may be indicated using various line types or may be indicated using color or other method of differentiation. Various types of demographic-based pain level state information may be selected using input 502 in some embodiments. Such demographics may include gender, age, race, income level, education, or any other type of demographic including dividing the respondents into those respondents that had higher pain level reactions from those with lower pain level reactions. A graph may be displayed indicating the various demographic groups, the line type or color for each group, the percentage of total respondents and/or absolute number of respondents for each group, and/or other information about the demographic groups. The pain level state information may be aggregated according to the demographic type selected. Thus, aggregation of the pain level state information is performed on a demographic basis so that pain level state information is grouped based on the demographic basis, for some embodiments. By way of example, a health care professional or researcher may be interested in observing the pain level state of a particular demographic group and may utilize a network 400 such as shown in FIG. 4.

Referring to FIG. 4 embodiments disclosed herein include a diagnostic system 202 for evaluating pain level states and the system may have an Internet or cloud connection 404 to assess patient pain level state information and a display such as 212 or 501 that may present the pain level assessment to the health care professional and/or to the patient. The diagnostic system 202 may be able to collect pain level state data from one or more patients as they interact with the system either at the site of a health care provider or at a remote location through portable wireless or wireline phone or a home computer system with Skype or some other visual connectivity. In some embodiments there may be multiple client computers that each collects pain level state data from patients as they interact with the diagnostic system.

As the pain level state data is collected, the diagnostic system may upload information to a server or analysis computer 401, based on the pain level state data from a plurality of patients. The diagnostic system 202 may communicate with the server 401 over the internet, intranet, some other computer network, or by any other method suitable for communication between two computers. In some embodiments, parts of the diagnostic system functionality may be embodied in the patient's computer. In some embodiments, computer 401 may interact with external databases or systems such as the PROMIS system as described herein.

The diagnostic system server 401 may have a connection to the internet directly or through cloud 404 to enable pain level state information to be received by the diagnostic system server. In some embodiments the pain level state information may include the patient pain level state information as well as pain level state information from other patients experiencing the same type of pain or the same type of illness. Further, the diagnostic system server may have a memory which stores instructions, data, help information and the like, and one or more processors attached to the memory wherein the one or more processors can execute instructions. The diagnostic system server may have a memory which stores instructions and one or more processors attached to the memory wherein the one or more processors can execute instructions. The memory may be used for storing instructions, for storing pain level state data, for system support, and the like. Server computer 401 may use its internet, or other computer communication method, to obtain pain level state information from various patients through various diagnostic systems including, in some embodiments, the PROMIS system. The diagnostic system server 401 may receive pain level state information collected from a plurality of patients from the diagnostic system, and may aggregate pain level state information on the plurality of patients.

The diagnostic system server 401 may process pain level state data or aggregated pain level state data gathered from a patient or a plurality of patients to produce pain level state information about the patient or a plurality of patients. In some embodiments, the diagnostic system server 401 may obtain pain level state information from computer 208 or robotic system 301. In this case the pain level state data may be analyzed by the diagnostic system 202 to produce pain level state information for uploading and possible viewing on display 212 or 501.

In some embodiments, the diagnostic system server 401 may receive or analyze data to generate aggregated pain level state information based on the pain level state data from the plurality of patients and may present aggregated pain level state information in a rendering on a display 212 or 501. In some embodiments, the analysis computer may be set up for receiving pain level state data collected from a plurality of patients, in a real-time or near real-time embodiment. In at least one embodiment, a single computer 401 may incorporate the client, server and analysis functionality. Patient pain level state data may be collected from the diagnostic systems 202 to form pain level state information on the patient or plurality of patients. Each diagnostic system 202 may include a computer program product embodied in a non-transitory computer readable medium.

Each of the above methods may be executed on one or more processors on one or more computer systems. Embodiments may include various forms of distributed computing, client/server computing, and cloud based computing. Further, it will be understood that for each flowchart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict processes, methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on.

A programmable apparatus that executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments disclosed herein are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. Accordingly, the spirit and scope of the claimed embodiments is not to be limited by the foregoing examples, but is to be understood in the broadest sense allowable by law.

1. A method for diagnosing pain state levels comprising the operations of:
collecting affective data including facial expressions;
collecting physiological measurement data;
analyzing collected affective and physiological data to determine pain state levels; and
communicating the pain state level information.

2. The method of claim 1 further including the operation of collecting speech data.

3. The method of claim 1 further including the operation of displaying the pain state level information.

4. The method of claim 1 wherein the operation of collecting physiological data includes at least one of: electrodermal activity; skin conductance; galvanic skin response; accelerometer readings; skin temperature; heart rate; heart rate variability;
blood pressure; electrocardiogram data; electroencephalogram data; and brain wave measurement.

5. The diagnostic system of claim 8 wherein the at least one physiological sensor includes a device for measuring analytes from at least one biomarker selected from sweat, saliva and blood.

6. The method of claim 5 wherein the device for measuring analytes includes one or more of measuring salivary cortisol, α-amylase (sAA), secretory IgA (sIgA), testosterone, or soluble fraction of receptor II of TNFα (sTNFαRII).

7. The method of claim 1 further including the operation of correlating collected affective and physiological data with data from a PROMIS system.

8. A diagnostic system for diagnosing a pain state level in a patient comprising:
at least one image capture device to record one or more facial data of the patient;
the facial data including one or more of: facial expressions; head, hand or body gestures; or body postures;
at least one physiological sensor associated with the patient to measure physiological data;
an interactive device in communication with the patient to permit the patient to input quantitative pain data into the diagnostic system;
a processor to generate a correlated data output from the one or more facial data, the physiological data, and the quantitative pain data, to determine the pain state level in the patient; and
an electronic display to communicate the determined patient pain state level to a health care provider and/or to the patient.

9. The diagnostic system according to claim 8 wherein the interactive device includes one or more of a keyboard, a mouse, a remote control, a motion sensor, a handheld portable phone, a computer, a paper questionnaire, or a camera sensor.

10. The diagnostic system according to claim 8 wherein the at least one physiological sensor includes one or more of: a galvanometer, photoplethysmography device, electromyography device, an accelerometer, detector for electrodermal activity, galvanic skin response measuring unit, skin temperature sensor, electrocardiogram, electroencephalogram, magnetic resonance imaging unit, and brain wave measurement unit.

11. The diagnostic system according to claim 8 wherein the at least one image capture device includes at least one of: a video camera, a webcam, a camera on a computer (including a laptop, a net-book, and a tablet), a still camera, a cell phone camera, a mobile device camera, a forward facing camera, a thermal imager, a charge coupled device, a three-dimensional camera, or a depth camera.

12. The diagnostic system according to claim 8 further including a speech capture device.

13. The diagnostic system according to claim 12 wherein the speech capture device includes a microphone.

14. The diagnostic system according to claim 8 wherein the electronic display includes: a computer display, a laptop screen, a net-book screen, a tablet computer screen, a cell phone display, a mobile device display, a remote with a display, a television, and a projector.

15. The diagnostic system according to claim 8 wherein the interactive device includes a PROMIS questionnaire in paper or electronic form.

16. A pain level diagnostic apparatus comprising:
a video screen viewed by a patient;
the video screen including an interactive pain assessment questionnaire;
at least one image capture device associated with the pain level diagnostic apparatus, the at least one image capture device positioned so as to be not visible to the patient and generating one or more facial data of the patient;
the facial data including one or more of: facial expressions; head, hand or body gestures; or body postures;
one or more physiological sensors associated with the patient to generate physiological data; and
a controller associated with the pain level diagnostic apparatus to record and analyze the one or more facial data and the physiological data and to correlate the recorded and analyzed data with a quantitative pain data input by the patient on the interactive pain assessment questionnaire to determine a pain state level of the patient;
whereby the determined pain state level may be communicated to a health care provider and/or to the patient.

17. The pain level diagnostic apparatus according to claim 16 further including a speech capture device.

18. The pain level diagnostic apparatus according to claim 16 wherein the video screen is a touchscreen.

19. The pain level diagnostic apparatus according to claim 16 wherein the one or more physiological sensors includes one or more of: a galvanometer, a photoplethysmography device, an electromyography device, an accelerometer, a detector for electrodermal activity, a galvanic skin response measuring unit, a skin temperature sensor, an electrocardiogram, an electroencephalogram, a magnetic resonance imaging unit, or a brain wave measurement unit.

20. The pain level diagnostic apparatus according to claim 16 wherein the at least one image capture device includes: a webcam, a camera on a computer (including a laptop, a net-book, and a tablet), a still camera, a cell phone camera, a mobile device camera, a forward facing camera, a thermal imager, a charge coupled device, a three-dimensional camera, a depth camera, or any other type of image capture apparatus that allows image data captured to be used by the pain level diagnostic apparatus.

21. The diagnostic system of claim 5 wherein the device for measuring analytes includes a device for measuring one or more of salivary cortisol, α-amylase (sAA), secretory IgA (sIgA), testosterone, or soluble fraction of receptor II of TNFα (sTNFαRII).

22. The diagnostic system of claim 8 wherein the facial expressions include at least one of: smiles and brow furrowing.

23. The pain level diagnostic apparatus according to claim 16 wherein the facial expressions include at least one of: smiles and brow furrowing.
